(19) United States
(12) Patent Application Publication
(10) Pub. No.: US 2025/0285740 A1
(43) Pub. Date: Sep. 11, 2025

(54) METHOD AND DEVICE FOR RECOGNIZING SURGICAL STAGE BASED ON VISUAL MULTIPLE MODALITY

(71) Applicant: HUTOM INC., Seoul (KR)

(72) Inventors: Bogyu PARK, Bucheon-si (KR); Hyeongyu CHI, Seoul (KR); Bokyung PARK, Seoul (KR); Jiwon LEE, Seoul (KR); Min Kook CHOI, Seoul (KR)

(73) Assignee: HUTOM INC., Seoul (KR)

(21) Appl. No.: 19/214,947

(22) Filed: May 21, 2025

Related U.S. Application Data
(63) Continuation of application No. PCT/KR2023/014457, filed on Sep. 22, 2023.

(30) Foreign Application Priority Data
Nov. 22, 2022 (KR) 10-2022-0157371

(51) Int. Cl. G16H 30/40; A61B 34/20; G06N 20/00

(52) U.S. Cl. CPC G16H 30/40; A61B 34/20; A61B 2034/2055; A61B 2034/2065; G06N 20/00

(57) ABSTRACT

The present disclosure relates to a method and device for recognizing a surgical stage based on visual multiple modality, and may include extracting a plurality of visual kinematics-based indices based on a surgical image including a plurality of frames corresponding to a plurality of surgical stages; obtaining first feature data for the surgical image, and obtain second feature data for the plurality of visual kinematics-based indices; obtaining third feature data by applying a fusion module learned to fuse data to the first feature data and the second feature data; and training a first artificial intelligence (AI) model to recognize each of the plurality of surgical stages based on the third feature data.

### CROSS-REFERENCE TO RELATED APPLICATIONS

The present application is a continuation of International Patent Application No. PCT/KR2023/014457, filed on Sep. 22, 2023, which is based upon and claims the benefit of priority to Korean Patent Application No. 10-2022-0157371 filed on Nov. 22, 2022. The disclosures of the above-listed applications are hereby incorporated by reference herein in their entirety.

### BACKGROUND

### 1. Technical Field

The present disclosure relates to a method and device for recognizing a surgical stage, and more particularly, the present disclosure relates to a method and device for recognizing a surgical stage based on visual multiple modality.

### 2. Description of Related Art

Accurate recognition and analysis of surgical stages can optimize surgical progress by enabling efficient communication and accurate situational judgment between the parties performing the surgery. In addition, accurate recognition of surgical stages can be useful when monitoring patients after surgery and when providing educational materials by classifying general surgical procedures.

However, recognition of surgical stages is a difficult task that includes the interaction of surgical instruments, organs included in the area where surgery is being performed, and activities such as camera cleaning and bleeding management. Conventionally, technologies that automatically recognize surgical stages by analyzing surgical images are studied, but there is a limitation in that they do not consider all of the above-described interactions related to surgical stages.

### SUMMARY

The present disclosure is to provide a method and device for recognizing a surgical step based on visual multiple modality.

Technical problems of the inventive concept are not limited to the technical problems mentioned above, and other technical problems not mentioned will be clearly understood by those skilled in the art from the following description.

In an aspect of the present disclosure, a method for recognizing a surgical stage based on visual multiple modality performed by a device may include extracting a plurality of visual kinematics-based indices based on a surgical image including a plurality of frames corresponding to a plurality of surgical stages; obtaining first feature data for the surgical image, and obtain second feature data for the plurality of visual kinematics-based indices; obtaining third feature data by applying a fusion module learned to fuse data to the first feature data and the second feature data; and training a first artificial intelligence (AI) model to recognize each of the plurality of surgical stages based on the third feature data.

In another aspect of the present disclosure, a device may include a memory configured to store at least one process for recognizing a surgical stage based on visual multiple modality; and a processor configured to perform an operation for recognizing the surgical stage as the process is executed, wherein the processor is configured to: extract a plurality of visual kinematics-based indices based on a surgical image including a plurality of frames corresponding to a plurality of surgical stages, obtain first feature data for the surgical image, and obtain second feature data for the plurality of visual kinematics-based indices, obtain third feature data by applying a fusion module learned to fuse data to the first feature data and the second feature data, and train a first artificial intelligence (AI) model to recognize each of the plurality of surgical stages based on the third feature data.

Furthermore, a computer program stored in a computer-readable recording medium for executing the present disclosure may be further provided.

Furthermore, a computer-readable recording medium recording a computer program for executing a method for executing the present disclosure may be further provided.

### BRIEF DESCRIPTION OF THE FIGURES

FIG. 1 is a schematic diagram of a system for implementing a method for recognizing a surgical stage based on visual multiple modality according to an embodiment of the present disclosure.

FIG. 2 is a block diagram illustrating a configuration of a device 100 for recognizing a surgical stage based on visual multiple modality according to an embodiment of the present disclosure.

FIG. 3 is a flowchart for describing a method for recognizing a surgical stage based on visual multiple modality performed by a device according to an embodiment of the present disclosure.

FIG. 4 is a diagram illustrating an overall structure of a method for recognizing a surgical stage based on visual multiple modality.

FIG. 5 is a diagram for describing a process of extracting feature data for a surgical image to recognize a surgical stage according to an embodiment of the present disclosure.

FIGS. 6A and 6B are diagrams for describing a process of extracting third feature data through a fusion module according to an embodiment of the present disclosure.

FIG. 7 is a diagram for describing a process of recognizing a surgical stage through a learned AI model by a device according to an embodiment of the present disclosure.

### DETAILED DESCRIPTION

In the drawings, the same reference numeral refers to the same element. This disclosure does not describe all elements of embodiments, and general contents in the technical field to which the present disclosure belongs or repeated contents of the embodiments will be omitted. The terms, such as “unit, module, member, and block” may be embodied as hardware or software, and a plurality of “units, modules, members, and blocks” may be implemented as one element, or a unit, a module, a member, or a block may include a plurality of elements.

Throughout this specification, when a part is referred to as being “connected” to another part, this includes “direct connection” and “indirect connection”, and the indirect connection may include connection via a wireless communication network. Furthermore, when a certain part “includes” a certain element, other elements are not excluded unless explicitly described otherwise, and other elements may in fact be included.

Furthermore, when a certain part “includes” a certain element, other elements are not excluded unless explicitly described otherwise, and other elements may in fact be included.

In the entire specification of the present disclosure, when any member is located “on” another member, this includes a case in which still another member is present between both members as well as a case in which one member is in contact with another member.

The terms “first,” “second,” and the like are just to distinguish an element from any other element, and elements are not limited by the terms.

The singular form of the elements may be understood into the plural form unless otherwise specifically stated in the context.

Identification codes in each operation are used not for describing the order of the operations but for convenience of description, and the operations may be implemented differently from the order described unless there is a specific order explicitly described in the context.

Hereinafter, operation principles and embodiments of the present disclosure will be described with reference to the accompanying drawings.

In this specification, the ‘device according to the present disclosure’ includes various devices that may perform computational processing and provide results to a user. For example, the device according to the present disclosure may include a computer, a server device, and a portable terminal, or may be in the form of one of them.

Here, the computer may include, for example, a notebook, desktop, laptop, tablet PC, slate PC, and the like. equipped with a web browser.

The server device is a server that communicates with an external device to process information, and may include an application server, a computing server, a database server, a file server, a game server, a mail server, a proxy server, and a web server.

The portable terminal may include, for example, a wireless communication device that ensures portability and mobility, such as a PCS (Personal Communication System), a GSM (Global System for Mobile communications), a PDC (Personal Digital Cellular), a PHS (Personal Handyphone System), a PDA (Personal Digital Assistant), an IMT (International Mobile Telecommunication)-2000, a CDMA (Code Division Multiple Access)-2000, a W-CDMA (W-Code Division Multiple Access), a WiBro (Wireless Broadband Internet) terminal, a smart phone, and all kinds of handheld-based wireless communication devices, and wearable devices such as a watch, a ring, a bracelet, an anklet, a necklace, glasses, contact lenses, or a head-mounted device (HMD).

In describing the present disclosure, a “user” may be a medical professional, such as a doctor, a nurse, a clinical pathologist, a medical imaging specialist, and the like, and may include a technician for repairing/controlling a medical device, but is not limited thereto.

In describing the present disclosure, “surgery” generally refers to a surgical treatment that incisions a skin or mucosa to perform a procedure for a disease or injury, and “surgical instrument” may generally refer to all tools used to perform a surgery.

In describing the present disclosure, “visual multiple modality” may mean multiple types of data that are visually implemented (e.g., surgical image data and visual kinematics-based indices, etc.).

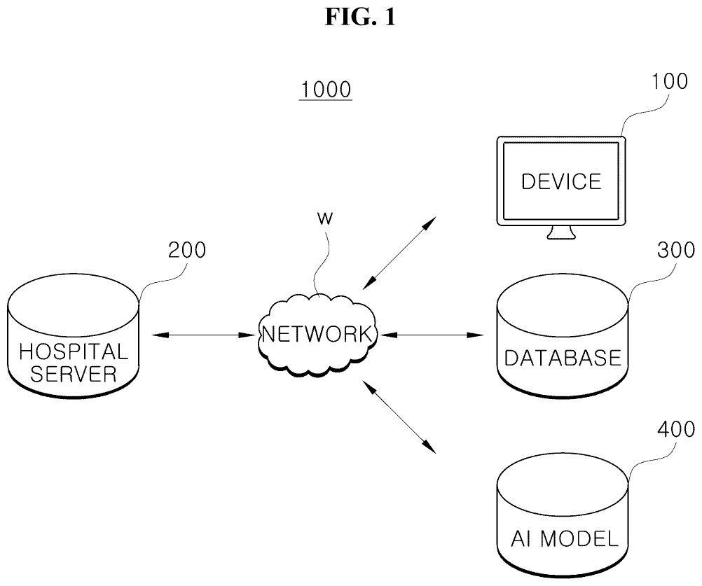

FIG. 1 is a schematic diagram of a system 1000 for implementing a method for recognizing a surgical stage based on visual multiple modality according to an embodiment of the present disclosure.

As shown in FIG. 1, a system 1000 for implementing a method for recognizing a surgical stage based on visual multiple modality may include a device 100, a hospital server 200, a database 300, and an AI model 400.

Here, although FIG. 1 illustrates that the device 100 is implemented in the form of a single desktop, it is not limited thereto. As described above, the device 100 may mean various types of devices or a group of devices in which one or more types of devices are connected.

The device 100, the hospital server 200, the database 300, and the artificial intelligence (AI) model 400 included in the system 1000 may communicate via a network W. Here, the network W may include a wired network and a wireless network. For example, the network may include various networks such as a local area network (LAN), a metropolitan area network (MAN), and a wide area network (WAN).

In addition, the network W may include the well-known World Wide Web (WWW). However, the network W according to the embodiment of the present disclosure is not limited to the networks listed above, and may include at least a part of a well-known wireless data network, a well-known telephone network, or a well-known wired/wireless television network.

The device 100 may obtain a surgical image including a plurality of frames corresponding to a plurality of surgical stages through the hospital server 200 or/and the database 300. However, this is merely an embodiment, and the device 100 may obtain a surgical image captured through a camera connected wirelessly/wirelessly to the device 100.

The device 100 may extract a plurality of visual kinematics-based indices based on the surgical image. The plurality of visual kinematics-based indices may include movement and interrelationship information of one or more surgical instruments included in the surgical image.

The device 100 may obtain third feature data that fuses first feature data for the surgical image and second feature data for the plurality of visual kinematics-based indices. Then, the device 100 may train the AI model 400 to recognize the surgical stage based on the third feature data.

The operation related thereto will be specifically described with reference to the drawings described below.

The hospital server 200 (e.g., a cloud server, etc.) may capture and store a surgical image of a patient. The hospital server 200 may transmit the surgical image stored in the device 100, the database 300, or the AI model 400.

The hospital server 200 may protect the personal information of the person in the surgical image by pseudonymizing or anonymizing the person in the surgical image. In addition, the hospital server may encrypt and store information related to the age/gender/height/weight/childbirth status of the patient who is the person in the surgical image input by the user.

The database 300 may store various feature data generated by the device 100 and one or more parameters/instructions for utilizing the AI model 400. Although FIG. 1 illustrates a case where the database 300 is implemented outside the device 100, the database 300 may also be implemented as a component of the device 100.

The AI model 400 is an artificial intelligence model learned to recognize a surgical stage through a surgical image. The AI model 400 may be learned to recognize a surgical stage through a data set constructed with feature data related to an actual surgical image. The learning method may include, but is not limited to, supervised training/unsupervised training. The detection data output through the AI model 400 may be stored in the database 300 or/and the memory of the device 100.

FIG. 1 illustrates a case where the AI model 400 is implemented outside the device 100 (e.g., cloud-based), but is not limited thereto, and may be implemented as a component of the device 100.

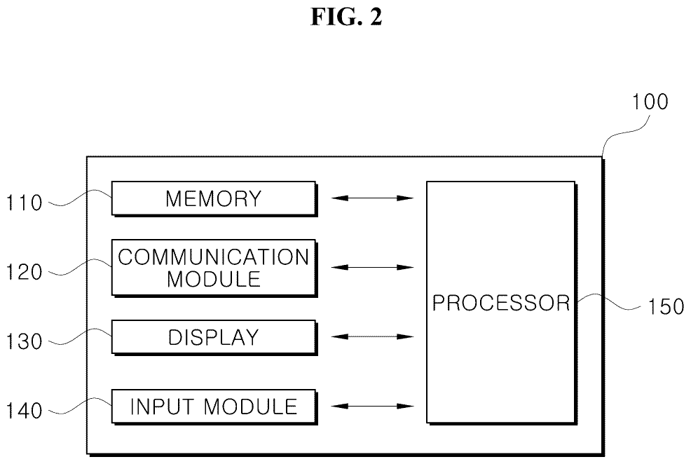

FIG. 2 is a block diagram illustrating a configuration of a device 100 for recognizing a surgical stage based on visual multiple modality according to an embodiment of the present disclosure.

As illustrated in FIG. 2, the device 100 may include a memory 110, a communication module 120, a display 130, an input module 140, and a processor 150. However, the present invention is not limited thereto, and the device 100 may have software and hardware configurations modified/added/omitted within a range obvious to those skilled in the art according to a required operation.

The memory 110 may store data supporting various functions of the device 100, at least one process or program for the operation of the processor 150, may store at least one process for recognizing a surgical stage based on visual multiple modality according to the present disclosure, may store input/output data (e.g., a full surgical image composed of multiple frames, one or more visual kinematics-based indices, etc.), and may store a plurality of application programs or applications run on the device, data for the operation of the device 100, and commands. At least some of these application programs may be downloaded from an external server via wireless communication.

The memory 110 may include at least one type of storage medium among a flash memory type, a hard disk type, an SSD type (Solid State Disk type), an SDD type (Silicon Disk Drive type), a multimedia card micro type, a card type memory (e.g., an SD or XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk.

In addition, the memory 110 may include a database that is separate from the device but connected by wire or wirelessly. That is, the database illustrated in FIG. 1 may be implemented as a component of the memory 110.

The communication module 120 may include one or more components that enable communication with an external device, and may include, for example, at least one of a broadcast reception module, a wired communication module, a wireless communication module, a short-range communication module, and a location information module.

The wired communication module may include various wired communication modules such as a Local Area Network (LAN) module, a Wide Area Network (WAN) module, or a Value Added Network (VAN) module, as well as various cable communication modules such as a Universal Serial Bus (USB), a High Definition Multimedia Interface (HDMI), a Digital Visual Interface (DVI), RS-232 (recommended standard232), power line communication, or a plain old telephone service (POTS).

The wireless communication module may include a wireless communication module that supports various wireless communication methods such as a WiFi module, a WiBro (Wireless broadband) module, GSM (Global System for Mobile Communication), CDMA (Code Division Multiple Access), WCDMA (Wideband Code Division Multiple Access), UMTS (Universal Mobile Telecommunications System), TDMA (Time Division Multiple Access), LTE (Long Term Evolution), 4G, 5G, and 6G.

The display 130 displays outputs information processed in the device 100 (e.g., a patient's surgical image, surgical stage recognition information corresponding to a specific frame constituting the surgical image, surgical skill score, etc.). For example, the display may display execution screen information of an application program (e.g., an application) running in the device 100, or UI (User Interface) and GUI (Graphical User Interface) information according to such execution screen information.

The input module 140 is for receiving information from a user, and when information is input through a user input module, the processor 150 may control the operation of the device 100 to correspond to the input information.

The input module 140 may include a hardware physical key (e.g., a button located on at least one of front, rear, and side of the device, a dome switch, a jog wheel, a jog switch, etc.). and a software touch key. As an example, the touch key may be formed as a virtual key, a soft key, or a visual key displayed on a touchscreen type display 130 through software processing, or as a touch key placed on a part other than the touchscreen. Meanwhile, the virtual key or visual key may be displayed on the touchscreen in various forms, and may be formed as, for example, a graphic, text, an icon, a video, or a combination thereof.

The processor 150 may control the overall operation and function of the device 100. Specifically, the processor 150 may be implemented as a memory that stores data for an algorithm for controlling the operation of components within the device 100 or a program that reproduces the algorithm, and at least one processor not shown that performs the operation using the data stored in the memory. At this time, the memory and the processor may be implemented as separate chips. Alternatively, the memory and the processor may be implemented as a single chip.

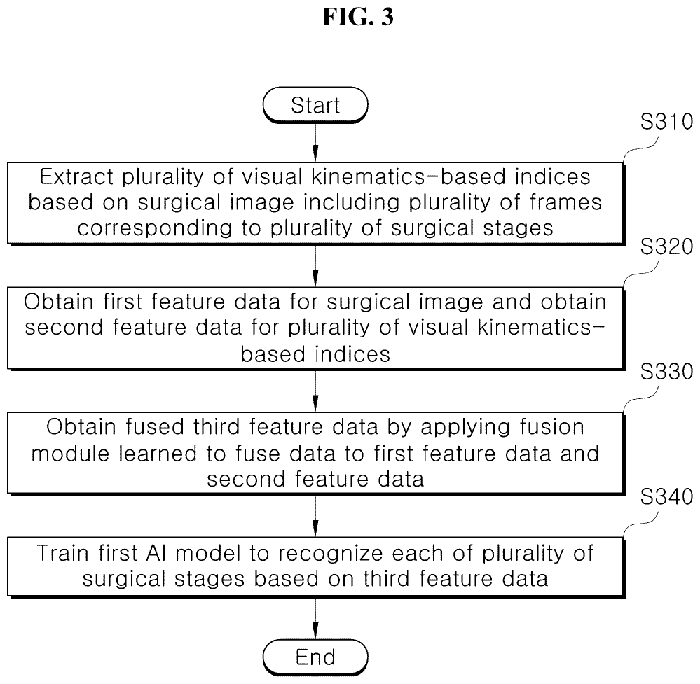

In addition, the processor 150 may control one or a combination of the components discussed above in order to implement various embodiments according to the present disclosure described in FIGS. 3 to 7 below on the device 100.

FIG. 3 is a flowchart for describing a method for recognizing a surgical stage based on visual multiple modality performed by a device according to an embodiment of the present disclosure.

The processor 150 of the device 100 may extract a plurality of visual kinematics-based indices based on a surgical image including a plurality of frames corresponding to a plurality of surgical stages (step S310).

Here, the plurality of visual kinematics-based indices may mean information indicating movement and interrelationship information of one or more surgical instruments included in the surgical image.

Specifically, the processor 150 may input a surgical image including a plurality of frames into a second AI model trained to perform a semantic segmentation algorithm to obtain semantic segmentation mask data. The processor 150 may extract a plurality of visual kinematics-based indices from the semantic segmentation mask data.

Here, the semantic segmentation algorithm means an algorithm that classifies all pixels of an image (or multiple frames/images constituting an image) into a pre-specified number of classes. The semantic segmentation algorithm may distinguish/classify/identify one or more body organs and surgical instruments that are surgical targets in an image (or multiple frames/images constituting an image), and mask the distinguished/classified/identified pixel areas.

Therefore, the semantic segmentation mask data may mean data that masks pixel areas classified as body organs and surgical instruments in an image or multiple frames/images constituting an image.

The processor 150 may extract multiple visual kinematics-based indices from semantic segmentation mask data corresponding to one or more surgical instruments included in a surgical image among the semantic segmentation mask data.

Specifically, the processor 150 may extract feature data related to a movement of the surgical instrument through semantic segmentation mask data corresponding to one or more surgical instruments. The device may extract the plurality of visual kinematics-based indices through the feature data related to the movement of the extracted surgical instrument.

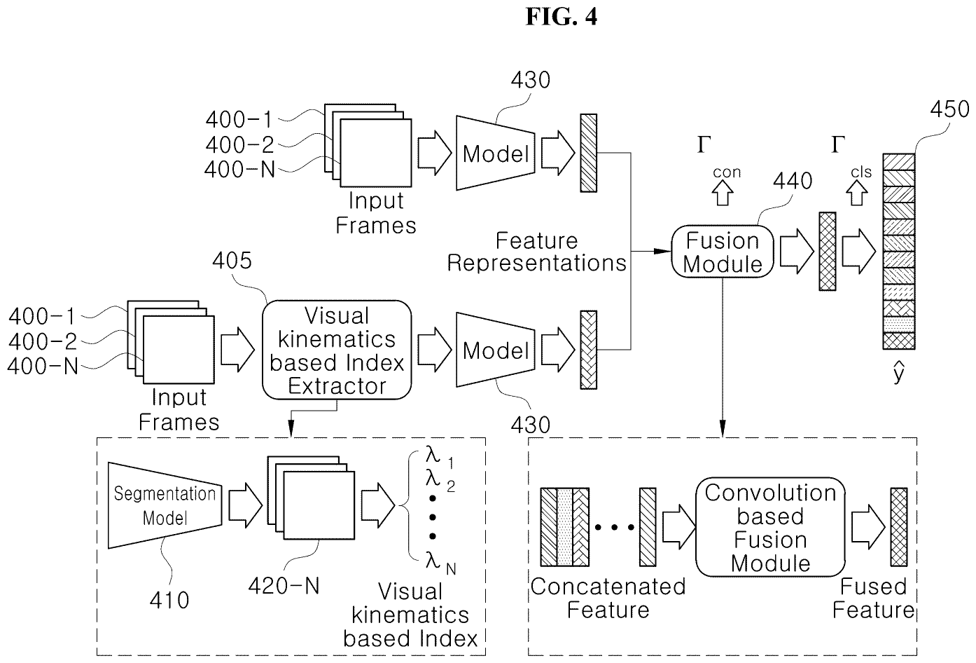

Referring to FIG. 4, the processor 150 may obtain a plurality of frames 400-1, 400-2, . . . and 400-N (N is a natural number greater than or equal to 1) representing a plurality of surgical stages constituting the surgical image. Here, the surgical image may be composed of frames representing the entire surgical process, but is not limited thereto.

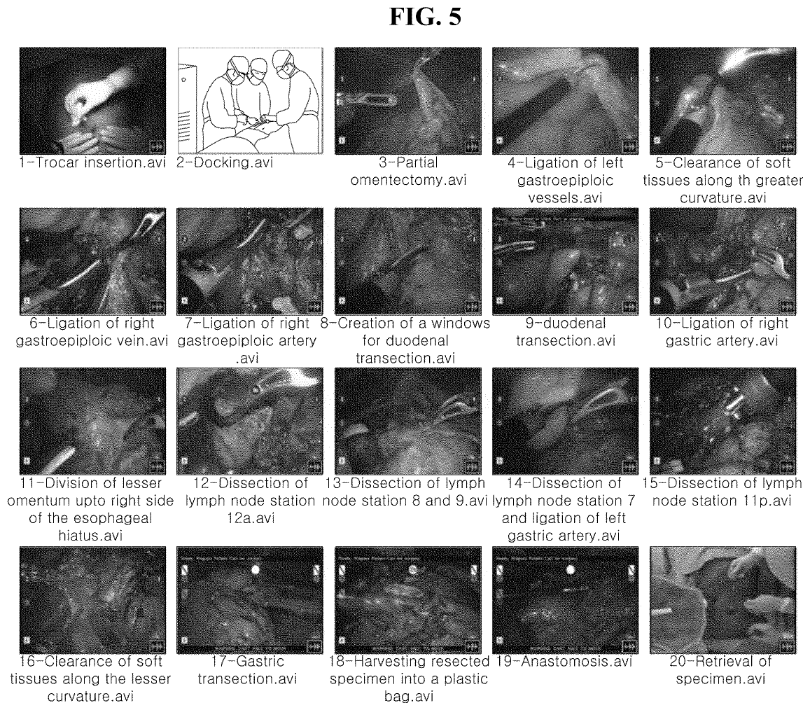

As an example, as illustrated in FIG. 5, the surgery may be divided into a plurality of processes (e.g., 20 processes), and the processor 150 may obtain images captured for each entire process. The plurality of frames 400-1, 400-2, . . . and 400-N illustrated in FIG. 4 may mean frames constituting images captured for each process.

The processor 150 may obtain a plurality of visual kinematic-based indices λ1, λ2 . . . , and λN by inputting the plurality of frames 400-1, 400-2, . . . and 400-N into a visual kinematic-based index extractor 405. Here, the visual kinematic-based index extractor 405 may include a second AI model 410 learned to perform a semantic segmentation algorithm.

The processor 150 may input multiple frames 400-1, 400-2, . . . and 400-N into the second AI model 410 to obtain semantic segmentation data 420-1, 420-2, . . . and 420-N corresponding to one or more surgical instruments. The processor 150 may obtain multiple visual kinematic-based indices λ1, λ2 . . . , and λN through the semantic segmentation data 420-1, 420-2, . . . and 420-N corresponding to one or more surgical instruments.

The visual kinematic-based indices may be classified into types based on the movement of the surgical instruments or the relationship between the surgical instruments. The movement of the surgical instrument may be measured as a path length, a speed, a centroid, a velocity, a bounding box, and an EOA (economy of area).

The measurement of the movement index (of the surgical instrument) may be implemented as in Equations 1 to 3.

$$PL = {\sum }_{t}^{T} \sqrt{{( D ( x , t ) )}^{2} + {( D ( y , t ) )}^{2}} , D ( x , t ) = {x}_{t} - {x}_{t - 1} , \qquad [ Equation 1 ]$$

$$s = \frac{PL}{T} , v ( x , t ) = \frac{{x}_{t} - {x}_{t - Δ}}{Δ} , \qquad [ Equation 2 ]$$

$$EOA = \frac{bw \times bh}{W \times H} \qquad [ Equation 3 ]$$

Here, PL represents the path length in the current time frame t, and T may represent the time range for computing the index. The path length may include a cumulative path length and a partial path length.

D (x, t) may measure the difference in the x-axis within the previous time frame and the current time frame. x and y may represent the center of gravity of the object within the frame. The center of gravity represents the average position value in the x and y coordinates of the semantic segmentation mask. s is the velocity for the time range T, and v may represent the velocity in the X or Y direction at the time interval Δ. bw and bh are the width and height of the bounding box, respectively, and W and H are the width and height of the image, respectively. The bounding box may include four values (top, left, box width, box height bx, by, bw, and bh).

The processor 150 may obtain first feature data for the surgical image and obtain second feature data for a plurality of visual kinematics-based indices (step S320).

Specifically, the processor 150 may obtain first feature data and second feature data by inputting the surgical image and each of the plurality of visual kinematics-based indices into the third AI model. Here, the third AI model may be configured based on at least one of a CNN (convolutional neural network) model and an LSTM (long short term memory) model.

The CNN model refers to the structure of a neural network model learned to perform a convolution operation, and the LSTM model refers to the structure of a neural network model designed to enable short-term/long-term memory by complementing the disadvantage of the RNN (recurrent neural network) model that may not remember information located far from the current output data.

Referring to FIG. 4, the processor 150 may input each of a surgical image (i.e., a plurality of frames constituting the surgical image) 400-1, 400-2, . . . and 400-N and a plurality of visual kinematic-based indices λ1, λ2 . . . , and λN into the third AI model 430 to obtain first feature data and second feature data.

FIG. 4 illustrates a case where the AI model input by each of the surgical image (i.e., multiple frames constituting the surgical image) 400-1, 400-2, . . . and 400-N and multiple visual kinematics-based indices λ1, λ2 . . . , and λN is the same. However, this is only an example, and the models input by the surgical image (i.e., multiple frames constituting the surgical image) 400-1, 400-2, . . . and 400-N and multiple visual kinematics-based indices λ1, λ2 . . . , and λN may be different.

As an example, the first feature data may include feature data related to a specific object (e.g., a body organ or a surgical instrument on which surgery is performed) in the multiple frames constituting the surgical image. The second feature data may include a movement pattern of the surgical instrument, and the like.

In another embodiment of the present disclosure, a surgical skill score of a user of at least one surgical instrument may be calculated based on a path and a movement pattern of at least one surgical instrument associated with a plurality of visual kinematics-based indices. The device may utilize a learned module to calculate the surgical skill based on the path and movement pattern of the predefined surgical instrument. The device may determine whether the user of the surgical instrument is a novice, an expert, or an expert based on the surgical skill score.

The processor 150 may obtain fused third feature data by applying a fusion module learned to fuse data to the first feature data and the second feature data (step S330).

Referring to FIG. 4, the processor 150 may obtain third feature data by applying the fusion module 440 to the first feature data and the second feature data. The processor 150 may concatenate each feature data and perform a convolution operation on the concatenated feature data to obtain third feature data 450.

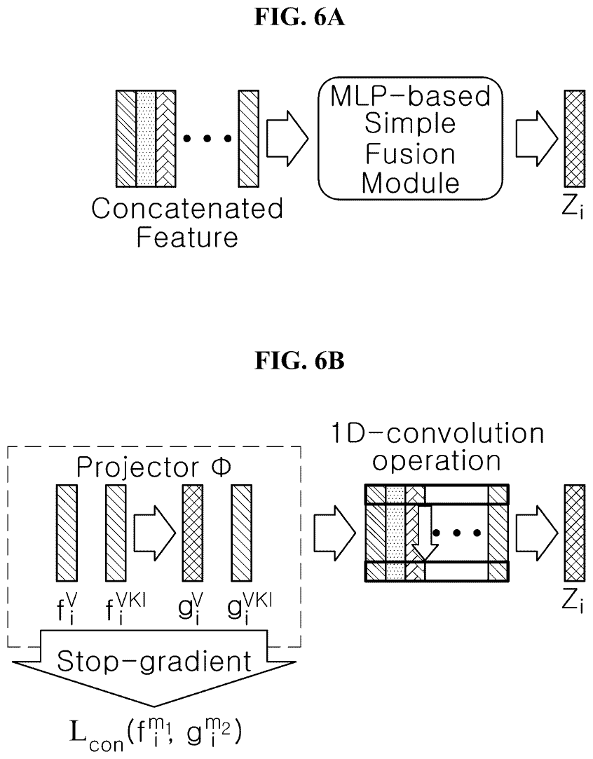

As an example, referring to FIG. 6A, the processor 150 may concatenate the first feature data and the second feature data. The processor 150 may obtain fused third feature data by applying the fusion module to the concatenated first feature data and the second feature data. Here, the fusion module may be configured based on a multi-layer perceptron (MLP).

As another example, referring to FIG. 6B, the fusion module may obtain enhanced data to enhance the interaction between the first feature data and the second feature data by applying a stop-gradient algorithm to the first feature data and the second feature data under the control of the processor 150. In addition, the fusion module may obtain third feature data by performing a convolution operation on the enhanced data under the control of the processor 150.

In order to apply the stop-gradient algorithm to the first feature data and the second feature data, the device may obtain a contrastive loss by utilizing Equations 4 to 6. The processor 150 may identify/learn the similarity between the feature data by utilizing the contrastive loss.

$${g}_{i}^{m} = φ ( {f}_{i}^{m} ) \qquad [ Equation 4 ]$$

$$D ( {a}_{i} , {b}_{i} ) = {( {\sum }_{j = 1}^{d} {\vert {a}_{i , j} - {b}_{i , j} \vert}^{p} )}^{1 / p} \qquad [ Equation 5 ]$$

$${L}_{con} ( {f}_{i}^{{m}_{1}} , {g}_{i}^{{m}_{2}} ) = \frac{1}{2} D ( {f}_{i}^{{m}_{1}} , stopgrad ( {g}_{i}^{{m}_{2}} ) ) + \frac{1}{2} D ( stopgrad ( {f}_{i}^{{m}_{1}} ) , {g}_{i}^{{m}_{2}} ) , \qquad [ Equation 6 ]$$

Here, fᵢ^{v} and fᵢ^{VKI} may each mean first feature data and second feature data. In addition, gᵢ^{m} having a different dimension and a different view may be generated through a projector composed of MLP. Each of ai and bi may mean feature data of a different view, p may represent the order of a vertical vector norm, and each of m1 and m2 may mean a surgical image and a visual kinematics-based index.

In addition, the processor 150 may perform a convolution operation on the enhanced data to enhance the interaction between the first feature data and the second feature data to obtain third feature data.

The processor 150 may train the first AI model to recognize each of the plurality of surgical stages based on the third feature data (step S340).

That is, when a specific frame of an arbitrary surgical image is input, the first AI model may be trained by the device to output information for the surgical stage indicated by the specific frame (i.e., information for distinguishing the surgical stage).

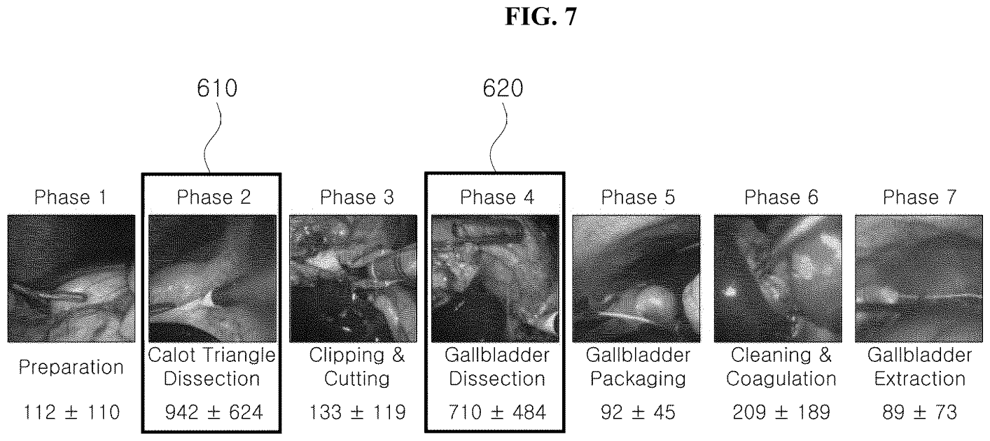

Referring to FIG. 7, the processor 150 may input a surgical image including frames indicating seven surgical stages into the first AI model. When a first frame 610 and a second frame 620 of the surgical image are played/selected, the first AI model may be trained to output calot triangle dissection and gallbladder dissection as the surgical stages corresponding to each frame.

Meanwhile, the disclosed embodiments may be implemented in the form of a recording medium storing commands executable by a computer. The instruction may be stored in the form of program codes, and when executed by a processor, the instruction may generate a program module to perform the operations of the disclosed embodiments. The recording medium may be implemented as a computer-readable recording medium.

The computer-readable recording medium includes all types of recording media that store instructions that may be deciphered by a computer. For example, there may be a ROM (Read Only Memory), a RAM (Random Access Memory), a magnetic tape, a magnetic disk, a flash memory, an optical data storage device, and the like.

The disclosed embodiments have been described with reference to the attached drawings as described above. Those skilled in the art to which the present disclosure pertains will understand that the present disclosure may be implemented in forms other than the disclosed embodiments without changing the technical idea or essential features of the present disclosure. The disclosed embodiments are exemplary and should not be construed as limiting.

### ADVANTAGEOUS EFFECTS

According to the above-described problem solving means of the present disclosure, a method and device for recognizing a surgical stage based on visual multi-modality can be provided.

According to the above-described problem solving means of the present disclosure, a method and device for training an artificial intelligence model for recognizing a surgical stage more accurately based on an image representing a surgical progress situation and information related to a surgical operation can be provided.

The effects of the present disclosure are not limited to the effects mentioned above, and other effects not mentioned can be clearly understood by those skilled in the art from the description.

What is claimed is:

1. A device comprising:
a memory configured to store at least one process for recognizing a surgical stage based on visual multiple modality; and
a processor configured to perform an operation for recognizing the surgical stage as the process is executed,
wherein the processor is configured to:
extract a plurality of visual kinematics-based indices based on a surgical image including a plurality of frames corresponding to a plurality of surgical stages,
obtain first feature data for the surgical image, and obtain second feature data for the plurality of visual kinematics-based indices,
obtain third feature data by applying a fusion module learned to fuse data to the first feature data and the second feature data, and
train a first artificial intelligence (AI) model to recognize each of the plurality of surgical stages based on the third feature data.

2. The device according to claim 1, wherein the processor is configured to:
when extracting the plurality of visual kinematics-based indices,
obtain semantic segmentation mask data by inputting the surgical image including the plurality of frames into a second AI model learned to perform a semantic segmentation algorithm, and
extract the plurality of visual kinematics-based indices from semantic segmentation mask data corresponding to one or more surgical instruments included in the surgical image among the semantic segmentation mask data.

3. The device according to claim 2, wherein the plurality of visual kinematics-based indices includes movement and interrelationship information of the one or more surgical instruments.

4. The device according to claim 3, wherein the processor is configured to:
when obtaining the first feature data and the second feature data,
obtain the first feature data and the second feature data by inputting each of the surgical image and the plurality of visual kinematics-based indices into a third AI model, and
wherein the third AI model includes at least one of a transformer, a convolutional neural network (CNN) model, and a long short term memory (LSTM) model.

5. The device according to claim 1, wherein the processor is configured to:
when obtaining the third feature data,
concatenate the first feature data and the second feature data, and
obtain the third feature data by applying the fusion module to the concatenated first feature data and the second feature data, and
wherein the fusion module includes a multi-layer perceptron-based fusion module.

6. The device according to claim 1, wherein the fusion module is configured to:
obtain enhanced data for enhancing an interaction between the first feature data and the second feature data by applying a stop-gradient algorithm to the first feature data and the second feature data, and
obtain the third feature data by performing a convolution operation on the enhanced data.

7. The device according to claim 1, wherein the processor is configured to:
calculates a surgical skill score of a user of the at least one surgical instrument based on a path and a movement pattern of the at least one surgical instrument related to the plurality of visual kinematics-based indices.

8. The device according to claim 1, wherein the first model learned based on the third feature data outputs information for the surgical stage indicated by the specific frame based on the specific frame of another surgical image being input by the device.

9. A method for recognizing a surgical stage based on visual multiple modality performed by a device, comprising:
extracting a plurality of visual kinematics-based indices based on a surgical image including a plurality of frames corresponding to a plurality of surgical stages;
obtaining first feature data for the surgical image, and obtain second feature data for the plurality of visual kinematics-based indices;
obtaining third feature data by applying a fusion module learned to fuse data to the first feature data and the second feature data; and
training a first artificial intelligence (AI) model to recognize each of the plurality of surgical stages based on the third feature data.

10. The method according to claim 9, wherein extracting the plurality of visual kinematics-based indices includes:
obtaining semantic segmentation mask data by inputting the surgical image including the plurality of frames into a second AI model learned to perform a semantic segmentation algorithm; and
extracting the plurality of visual kinematics-based indices from semantic segmentation mask data corresponding to one or more surgical instruments included in the surgical image among the semantic segmentation mask data.

11. The method according to claim 10, wherein the plurality of visual kinematics-based indices includes movement and interrelationship information of the one or more surgical instruments.

12. The method according to claim 11, wherein obtaining the first feature data and the second feature data includes:
obtaining the first feature data and the second feature data by inputting each of the surgical image and the plurality of visual kinematics-based indices into a third AI model, and
wherein the third AI model includes at least one of a transformer, a convolutional neural network (CNN) model, and a long short term memory (LSTM) model.

13. The method according to claim 9, wherein obtaining the third feature data includes:
concatenating the first feature data and the second feature data; and
obtaining the third feature data by applying the fusion module to the concatenated first feature data and the second feature data, and
wherein the fusion module includes a multi-layer perceptron-based fusion module.

14. The method according to claim 9, wherein the fusion module is configured to:
obtain enhanced data for enhancing an interaction between the first feature data and the second feature data by applying a stop-gradient algorithm to the first feature data and the second feature data; and
obtain the third feature data by performing a convolution operation on the enhanced data.

15. The method according to claim 9, further comprising:
calculating a surgical skill score of a user of the at least one surgical instrument based on a path and a movement pattern of the at least one surgical instrument related to the plurality of visual kinematics-based indices.
